(19) United States
(12) Patent Application Publication
(10) Pub. No.: US 2017/0240978 A1
(43) Pub. Date: Aug. 24, 2017

(54) SNP MARKER AS WELL AS USE AND DETECTION METHOD THEREOF

(71) Applicant: INSTITUTE OF APICULTURAL RESEARCH, CHINESE ACADEMY OF AGRICULTURAL SCIENCES, Beijing (CN)

(72) Inventors: Xiao CHEN, Beijing (CN); Wei SHI, Beijing (CN); Chao CHEN, Beijing (CN); Zhiguang LIU, Beijing (CN); Huihua WANG, Beijing (CN); Haikun GUO, Beijing (CN)

(73) Assignee: INSTITUTE OF APICULTURAL RESEARCH, CHINESE ACADEMY OF AGRICULTURAL SCIENCES, Beijing (CN)

(21) Appl. No.: 15/411,654

(22) Filed: Jan. 20, 2017

(30) Foreign Application Priority Data
Feb. 22, 2016 (CN) 201610096189.8

(51) Int. Cl. C12Q 1/68

(52) U.S. Cl. CPC C12Q 1/6888; C12Q 2600/156

(57) ABSTRACT

The present invention relates to the field of biotechnology, and in particular to SNP markers as well as use and detection method thereof. The present invention employs whole genome sequencing to conduct whole genome sequencing on a large number of individuals of Apis mellifera sinisxinyuan, and obtains 21 pieces of SNP information specific in Apis mellifera sinisxinyuan, which become marker SNP sites for identifying Apis mellifera sinisxinyuan. The SNP sites provided by the present invention, which are specific in Apis mellifera sinisxinyuan, play important roles in the differentiation of western honey bee in China from the western honey bees in other regions, and in the adaptive evolution of western honey bee in China to the local environment.

### CROSS REFERENCE OF RELATED APPLICATION

The present application claims the priority of China Patent Application No. 201610096189.8, filed with the Patent Office of China on Feb. 22, 2016, titled “SNP MARKER AS WELL AS USE AND DETECTION METHOD THEREOF”, the contents of which are incorporated herein by reference in its entirety.

### FIELD OF THE INVENTION

The present invention relates to the field of biotechnology, in particular to SNP markers as well as use and detection method thereof.

### BACKGROUND OF THE INVENTION

With a long history of apiculture, the germplasm resources of honey bees are rich in China; however, for a long period of time, only eastern honey bees rather than native western honey bees have been found in the territory of China. Since the collection ability of the eastern honey bees is slightly inferior to that of the western honey bees, an increasing number of Chinese beekeepers begin to breed the western honey bees with the introduction thereof, and thus the germplasm resources of native bees are threatened in China. If native western honey bees can be found in the territory of China, it will be of critical significance to the germplasm resources of honey bees in China. As the director of Chinese Honey Bee Germplasm Resources Committee, Dr. Shi Wei along with the staff of Xinjiang Autonomous Region apiculture management station have been engaged in strengthening the protection work of the original Yili dark bee of Xinjiang, and with several years of efforts, they discovered a natural population (Apis mellifera sinisxinyuan) for the first time in the territory of Yili, Xinjiang of China, which has a differentiation of at least 132,000 years from the other western honey bee subspecies currently known internationally, demonstrating that China is also an origin of western honey bees and terminating the history that there is no natural distribution of western honey bees in China, which is a great breakthrough in the aspects of livestock and poultry resources research. Apis mellifera sinisxinyuan has large body, good performance for wintering, strong stress resistance, outstanding ability of mite resistance and strong oviposition ability of the queen which can maintain a strong group and huge collection ability of the bee colony; Apis mellifera sinisxinyuan can perform both as a new species to be popularized and as a breeding material for further in-depth breeding research, which possesses good development prospects.

The discovery of western honey bee in China has a great impact at home and abroad. In order to more deeply protect the Apis mellifera sinisxinyuan and to effectively employ the Apis mellifera sinisxinyuan for services of honey bee breeding in China, with the supports of “Bee industry technology system”, “Conservation of species resources” and other projects, we have achieved a breakthrough in the research work on the Apis mellifera sinisxinyuan, and identified 21 SNP sites specific in Apis mellifera sinisxinyuan using the whole genome sequencing technology.

The whole genome sequencing refers to sequencing all genes in the genome of an organism to determine the DNA base sequence thereof. The whole genome sequencing has a wide coverage and can detect all of the genetic information in genome of an individual with high accuracy. Each individual inherits the DNA genetic information from parents at the beginning of a fertilized egg, and the genetic information is carried for the whole life and hardly changes. The whole genome sequencing is a process performed by applying a new generation high-throughput DNA sequencer for individual whole genome sequencing with a coverage rate of 10-20 times, and then comparing with the precise map of the genome of the same species to obtain the complete whole genome sequence of the individual and thus deciphering all the genetic information of the individual. For the whole-genome sequenced individuals, by means of sequence alignment, a large amount of single nucleotide polymorphism (SNP) sites specific to a particular species (strain) can be found.

Currently, researchers typically employ the SNP site information of mitochondrial DNA to identify the bee species information. The detection method of a SNP site of mitochondrial DNA is as follows: firstly extracting individual genomic DNA, and then performing PCR amplification of mitochondrial DNA and in turn sequencing to obtain the SNP information of mitochondrial DNA sequence.

The currently existing technical problems:

(1) little information of SNP sites;

(2) low accuracy of the bee species identification;

(3) one-sidedness.

### SUMMARY OF THE INVENTION

In view of this, the present invention provides SNP markers as well as use and detection method thereof. The present invention employs the whole genome sequencing to perform whole genome sequencing on a large number of individuals of Apis mellifera sinisxinyuan and obtains 21 pieces of SNP information specific in Apis mellifera sinisxinyuan, which become marker SNP sites for identifying Apis mellifera sinisxinyuan.

In order to achieve the above inventive object, the present invention provides the following technical solutions:

The present invention provides a SNP marker, selected from one or more of:

| | | |
|---|---|---|
| NW_003378074.1 | Position 935827 | SNP Information T; |
| NW_003378158.1 | Position 846767 | SNP Information A; |
| NW_003378051.1 | Position 292887 | SNP Information C; |
| NW_003378051.1 | Position 512953 | SNP Information C; |
| NW_003378077.1 | Position 1503304 | SNP Information T; |
| NW_003378131.1 | Position 693133 | SNP Information T; |
| NW_003378131.1 | Position 860330 | SNP Information T; |
| NW_003378091.1 | Position 420520 | SNP Information A; |
| NW_003378085.1 | Position 1765707 | SNP Information A; |
| NW_003378085.1 | Position 1775747 | SNP Information T; |
| NW_003378085.1 | Position 2595617 | SNP Information T; |
| NW_003378054.1 | Position 1502972 | SNP Information T; |
| NW_003378088.1 | Position 3767987 | SNP Information A; |
| NW_003378088.1 | Position 3816341 | SNP Information C; |
| NW_003378088.1 | Position 3914549 | SNP Information A; |
| NW_003378088.1 | Position 4363281 | SNP Information A; |
| NW_003377992.1 | Position 391791 | SNP Information T; |
| NW_003378010.1 | Position 550054 | SNP Information A; |
| NW_003378027.1 | Position 356859 | SNP Information A; |
| NW_003378033.1 | Position 211517 | SNP Information T; |
| NW_003377995.1 | Position 360953 | SNP Information G. |

The present invention also provides use of the SNP marker in identification of a species, wherein the species is Apis mellifera sinisxinyuan.

The present invention also provides use of the SNP marker in molecular marker-assisted breeding of Apis mellifera sinisxinyuan.

The present invention also provides use of the SNP marker in genetic diversity of species resource.

The present invention also provides use of the SNP marker in adaptive evolution of species.

The present invention also provides a primer pair for detecting the SNP marker.

The present invention also provides a kit for detecting the SNP marker, which comprises the primer pair.

The present invention also provides a method of identifying Apis mellifera sinisxinyuan with the SNP marker, which comprises:

step 1: obtaining the DNA of a test species, amplifying with the primer pair of claim 6 to obtain an amplification product;

step 2: subjecting the amplification product obtained in step 1 to single nucleotide polymorphism detection, wherein the detection criterion is as follows:

| | | |
|---|---|---|
| NW_003378074.1 | Position 935827 | SNP Information T; |
| NW_003378158.1 | Position 846767 | SNP Information A; |
| NW_003378051.1 | Position 292887 | SNP Information C; |
| NW_003378051.1 | Position 512953 | SNP Information C; |
| NW_003378077.1 | Position 1503304 | SNP Information T; |
| NW_003378131.1 | Position 693133 | SNP Information T; |
| NW_003378131.1 | Position 860330 | SNP Information T; |

| | | |
|---|---|---|
| NW_003378091.1 | Position 420520 | SNP Information A; |
| NW_003378085.1 | Position 1765707 | SNP Information A; |
| NW_003378085.1 | Position 1775747 | SNP Information T; |
| NW_003378085.1 | Position 2595617 | SNP Information T; |
| NW_003378054.1 | Position 1502972 | SNP Information T; |
| NW_003378088.1 | Position 3767987 | SNP Information A; |
| NW_003378088.1 | Position 3816341 | SNP Information C; |
| NW_003378088.1 | Position 3914549 | SNP Information A; |
| NW_003378088.1 | Position 4363281 | SNP Information A; |
| NW_003377992.1 | Position 391791 | SNP Information T; |
| NW_003378010.1 | Position 550054 | SNP Information A; |
| NW_003378027.1 | Position 356859 | SNP Information A; |
| NW_003378033.1 | Position 211517 | SNP Information T; |
| NW_003377995.1 | Position 360953 | SNP Information G. |

In some particular embodiments, in the method of identifying Apis mellifera sinisxinyuan with the SNP marker as provided in the present invention, if the species to be tested satisfies 95% or more of the detection criterion, the species to be tested is Apis mellifera sinisxinyuan; if the species to be tested does not satisfy at least two of the detection criteria, the species to be tested is not Apis mellifera sinisxinyuan.

The present invention employs the whole genome sequencing to conduct the whole genome sequencing on a large number of individuals of Apis mellifera sinisxinyuan, and obtains 21 pieces of SNP information specific in Apis mellifera sinisxinyuan, which become marker SNP sites for identifying Apis mellifera sinisxinyuan A. The SNP sites provided by the present invention, which are specific in Apis mellifera sinisxinyuan, play important roles in the differentiation of the western honey bees in China from the other western honey bees in other regions, and in the adaptive evolution of western honey bees in China to the local environment.

### DESCRIPTION OF DRAWINGS

In order to illustrate the examples of the present invention or the technical solutions in the prior art more clearly, the drawings which are required for use in the examples or the prior art descriptions will be briefly described below.

FIG. 1 shows the graph of principal component analysis on Apis mellifera sinisxinyuan and other major bee species using morphological identification data. It can be seen from the figure that Apis mellifera sinisxinyuan and Apis mellifera mellifera are clustered together and can not be distinguished from each other.

FIG. 2 shows the phylogenetic tree constructed using the mitochondrial DNA sequencing data on Apis mellifera sinisxinyuan and other major bee species. It can be seen in the figure that Apis mellifera sinisxinyuan and Apis mellifera mellifera are clustered together and can not be distinguished from each other.

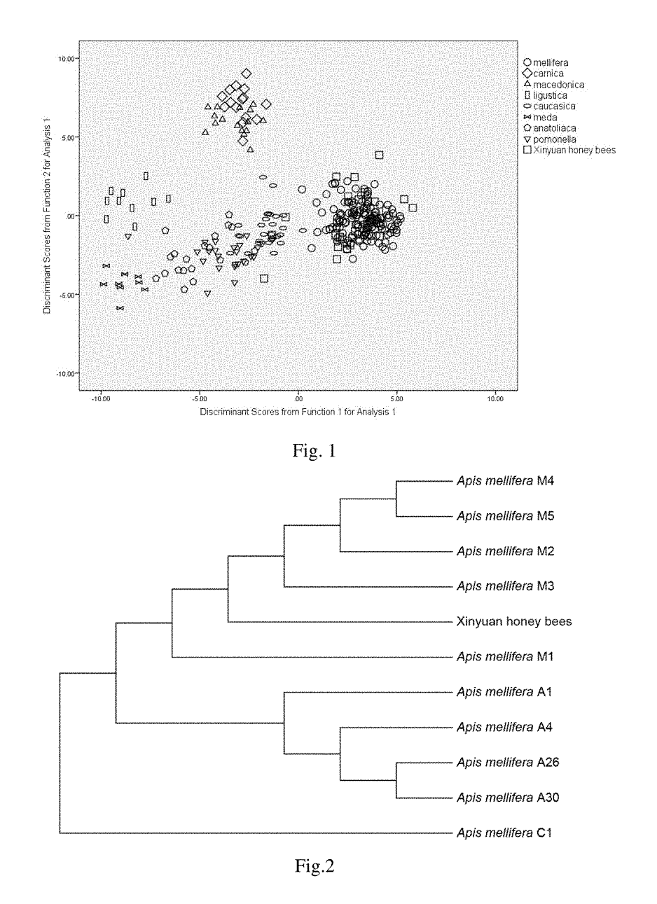

### DETAILED DESCRIPTION OF EMBODIMENTS

The present invention discloses SNP makers as well as use and detection method thereof, and those skilled in the art may use the content herein for reference to improve the technological parameters appropriately to achieve it. It should be particularly noted that all the similar substitutions and alterations will be apparent to those skilled in the art, and are deemed to be included in the present invention. The method and use of the present invention have been described by way of preferred embodiments, and it will be apparent to the related personnel that the method and use described herein may be altered or appropriately modified and combined to achieve and apply the technology of the present invention without departing from the content, spirit and scope of the present invention.

1. Sample collection. Collecting living honey bee samples and immediately putting them into 75% ethanol for storage. Furthermore, for the sample preservation, in addition to 75% ethanol for storage, ethanol with higher purity (90%), or liquid nitrogen, dry ice and other low-temperature preservation methods can also be used.

2. Extracting high-quality DNA of the samples.

3. Subjecting the DNA samples to Illumina high-throughput sequencing to obtain the raw data of DNA sequences. Any high-throughput sequencing platforms can be used for DNA high-throughput sequencing; in addition to the Illumina platform described above, SOLiD platform, 454 sequencing and the like can also be used.

4. Filtering out the low-quality sequences. The rules for filtering include: 1) the number of terminal “N” should be less than or equal to 10% of the sequence length; 2) the number of base with sequencing quality lower than 5 should be no more than 50% of the sequence length.

5. Performing sequence alignment using BWA software by taking Apis mellifera genome in the NCBI public database as the reference genome (apiMe14.5), wherein the alignment parameter is “-t -k 32 -M -R”. The latest version of the reference genome is apiMel4.5, and the reference genome of updated version can be employed after their publication.

6. Obtaining the SNP genotypes of a population using the SAMtools' mpileup program, and filtering the obtained genotypes to obtain the final results. The rules for filtering are: 1) the quality value should be no less than 20; 2) SNPs within 5 bases from a sequence gap should be filtered out; 3) the sequencing depth should be greater than or equal to 4, and less than or equal to 1000; 4) SNP sites with 3 or more genotypes are removed; the programs for obtaining SNP genotypes can be any programs that is capable of performing variation test, including the above-described SAMtools, and GATK, CLC and other programs.

7. The genotypes based on the 21 SNP sites of each sample to be tested were detected. If all of the samples to be tested satisfy more than 95% of the criteria of the following (1)-(21), the honey bee population to be tested is a candidate of Apis mellifera sinisxinyuan, i.e. if any 20 or 21 SNPs of the 1st to 21st SNPs are satisfied, the population is Apis mellifera sinisxinyuan.

(1) NW_003378074.1, position 935827, SNP information T;

(2) NW_003378158.1, position 846767, SNP information A;

(3) NW_003378051.1, position 292887, SNP information C;

(4) NW_003378051.1, position 512953, SNP information C;

(5) NW_003378077.1, position 1503304, SNP information T;

(6) NW_003378131.1, position 693133, SNP information T;

(7) NW_003378131.1, position 860330, SNP information T;

(8) NW_003378091.1, position 420520, SNP information A;

(9) NW_003378085.1, position 1765707, SNP information A;

(10) NW_003378085.1, position 1775747, SNP information T;

(11) NW_003378085.1, position 2595617, SNP information T;

(12) NW_003378054.1, position 1502972, SNP information T;

(13) NW_003378088.1, position 3767987, SNP information A;

(14) NW_003378088.1, position 3816341, SNP information C;

(15) NW_003378088.1, position 3914549, SNP information A;

(16) NW_003378088.1, position 4363281, SNP information A;

(17) NW_003377992.1, position 391791, SNP information T;

(18) NW_003378010.1, position 550054, SNP information A;

(19) NW_003378027.1, position 356859, SNP information A;

(20) NW_003378033.1, position 211517, SNP information T;

(21) NW_003377995.1, position 360953, SNP information G.

If there are at least two of the tested samples that do not satisfy all the criteria of (1) to (21), the honey bee population to be tested is not a candidate of Apis mellifera sinisxinyuan.

The present invention discloses a method of identifying Apis mellifera sinisxinyuan species using 21 SNPs. The method of assisting in the identification of whether or not a honey bee is Apis mellifera sinisxinyuan provided by the present invention comprises the following steps of: the genotype based on the 21 SNP sites of the honey bee to be tested is detected; if 95% or more criteria of (1)-(21) is satisfied, the honey bee to be tested is a candidate of Apis mellifera sinisxinyuan; if ≧95% criteria of (1)-(21) is not satisfied, the honey bee to be tested is not a candidate of Apis mellifera sinisxinyuan A. The present invention has important value for identification of the bee species germplasm resources of Apis mellifera sinisxinyuan.

The advantageous effects of the present invention lies in that:

(1) The present invention employs the whole genome sequencing technology, of which the sequencing depth covers the gene sequences of the whole genome of the honey bee, and can comprehensively and accurately detect the SNP information of Apis mellifera sinisxinyuan; therefore, the 21 SNPs for identifying Apis mellifera sinisxinyuan listed in the present invention have the characteristics of comprehensiveness and accuracy.

(2) The specific SNP sites for identifying Apis mellifera sinisxinyuan listed in the present invention play important roles in the differentiation of western honey bees in China from the western honey bees in other regions, and in the adaptive evolution of the western honey bee in China to the local environment.

All of the raw materials and reagents used in the SNP markers as well as use and detection method thereof provided by the present invention are commercially available.

The present invention is further illustrated in conjunction with the following examples:

### Example 1

(1) Honey bee samples were collected and DNA was extracted; samples with the OD value of the DNA being 1.8-2.0, content over 1.5 μg were considered to be qualified.

(2) A library was constructed with the qualified DNA samples: The DNA samples tested to be qualified were broken randomly into fragments with a length of 350 bp via a Covaris crusher. TruSeq Library Construction Kit was employed to construct the library and the reagents and consumables recommended in the manual were used strictly. DNA fragments were subjected to end-repair, tail addition, sequencing adaptor addition, purification, PCR amplification and other steps to accomplish the preparation of the whole library. The well-constructed library was sequenced by illumina HiSeq.

(3) Library inspection: After the library was constructed, Qubit2.0 was used first for preliminary quantification and the library was diluted to 1 ng/μl; subsequently, the insert size of the library was detected with Agilent 2100. After the insert size met the expectation, the effective concentration of the library was accurately qualified by Q-PCR method (effective concentration of the library>2 nM) to ensure the quality of the library.

(4) Sequencing on machine: With library inspection qualified, illumina HiSeq sequencing was conducted according to the effective concentration of the library and requirements of data output.

(5) Quality control: Sequenced Reads or raw reads obtained by sequencing contain low-quality reads with adaptors. In order to ensure the quality of information analysis, raw reads must be filtered to obtain clean reads, and all of the following analyses were based on the clean reads. Data processing steps are as follows:

a. removing paired-end reads with adaptors;

b. such paired-end reads need to be removed when the content of N contained in the single-end sequencing read exceeds 10% of the read length;

c. such paired-end reads need to be removed when the number of low-quality (Q<=5) base contained in the single-end sequencing read exceeds 50% of the read length.

(6) Sequence alignment:

Sequence (clean reads) alignment was conducted with the BWA software, and default values were adopted for all parameters except “-t-k 32-M-R”. With Amel 4.5 (derived from NCBI) taken as the reference genome, the bam files obtained from alignment were sorted with the SAMtools software and the duplicated sequences were removed.

(7) SNP detection: After the bam files were obtained, SNP detection was performed. SNP (single nucleotide polymorphism) mainly refers to DNA sequence polymorphism caused by a single nucleotide variation on genomic level, including transition, transversion, etc. of a single base. SAMTOOLS (mpileup-m2-F 0.002-d 1000) was used for individual SNP detection. In order to reduce the error rate of SNP detection, the following criteria were selected for filtering:

a. the support number of SNP reads is no less than 4;

b. the quality value (MQ) of SNPs is no less than 20;

(8) SNP annotation: ANNOVA is an efficient software tool that uses the latest information to annotate gene variations detected from multiple genomes. ANNOVAR can perform gene-based annotation, region-based annotations, filter-based annotation, and other functionalities as long as the chromosomes where the variation is located, start sites, stop sites, reference nucleotides and variant nucleotides are given. In view of the powerful annotation capability and international acceptance of ANNOVAR, it was used in annotating SNP detection results.

(9) Screening and aligning to obtain the SNPs specific to Apis mellifera sinisxinyuan: the SNPs obtained in the present study were aligned with the genomic SNP information of several major bee species of A. m. mellifera, A. m. carnica, A. m. ligustica, A. m. anatoliaca, A. m. scutellata and A. cerana, and the SNPs which were located in the gene coding regions, were non-synonymous mutations, and appeared only in Apis mellifera sinisxinyuan Apis were screened out, which are the SNPs specific to Apis mellifera sinisxinyuan.

The SNPs specific to Apis mellifera sinisxinyuan were obtained via screening and aligning, which are specifically listed below:

(1) NW_003378074.1, position 935827, SNP information T;

(2) NW_003378158.1, position 846767, SNP information A;

(3) NW_003378051.1, position 292887, SNP information C;

(4) NW_003378051.1, position 512953, SNP information C;

(5) NW_003378077.1, position 1503304, SNP information T;

(6) NW_003378131.1, position 693133, SNP information T;

(7) NW_003378131.1, position 860330, SNP information T;

(8) NW_003378091.1, position 420520, SNP information A;

(9) NW_003378085.1, position 1765707, SNP information A;

(10) NW_003378085.1, position 1775747, SNP information T;

(11) NW_003378085.1, position 2595617, SNP information T;

(12) NW_003378054.1, position 1502972, SNP information T;

(13) NW_003378088.1, position 3767987, SNP information A;

(14) NW_003378088.1, position 3816341, SNP information C;

(15) NW_003378088.1, position 3914549, SNP information A;

(16) NW_003378088.1, position 4363281, SNP information A;

(17) NW_003377992.1, position 391791, SNP information T;

(18) NW_003378010.1, position 550054, SNP information A;

(19) NW_003378027.1, position 356859, SNP information A;

(20) NW_003378033.1, position 211517, SNP information T;

(21) NW_003377995.1, position 360953, SNP information G.

The above SNPs are specific to Apis mellifera sinisxinyuan. Any samples of which the SNP information contained in the genome complies with 95% or more of the SNP information in the list above is Apis mellifera sinisxinyuan, otherwise is not.

### Example 2

The testing method of identifying Apis mellifera sinisxinyuan from other bee species through the SNP sites in Example 1 refers to the method of Example 1. The test results are shown in Table 1:

TABLE 1
Identification results of SNP genotypes

| | | | | | | | | | Apis |
|---|---|---|---|---|---|---|---|---|---|
| | | | | | | Chinese | | | mellifera |
| | | | Italian | Carniolan | European | honey bee | Zhejiang | | sinisxinyuan |
| SNP | | | bee | bee | dark bee | Apis | royal | Northeastern | (Xinyuan |
| genotype | Accession | Locus | Ligustica | Carnica | mellifera | cerana | jelly bee | dark bee | honey bee) |
| 1 | NW_003378074.1 | 935827 | C | C | C | C | C | C | Y |
| 2 | NW_003378158.1 | 846767 | — | G | G | G | G | G | A |
| 3 | NW_003378051.1 | 292887 | — | G | G | G | G | G | S |
| 4 | NW_003378051.1 | 512953 | — | T | T | T | T | T | Y |
| 5 | NW_003378077.1 | 1503304 | G | G | G | G | G | G | R |
| 6 | NW_003378131.1 | 693133 | — | C | — | C | C | C | Y |
| 7 | NW_003378131.1 | 860330 | — | C | C | C | C | C | Y |
| 8 | NW_003378091.1 | 420520 | — | G | G | G | G | G | R |
| 9 | NW_003378085.1 | 1765707 | — | G | G | G | G | G | A |
| 10 | NW_003378085.1 | 1775747 | C | C | — | C | C | C | T |
| 11 | NW_003378085.1 | 2595617 | C | C | C | — | C | C | Y |
| 12 | NW_003378054.1 | 1502972 | — | G | G | G | G | G | K |
| 13 | NW_003378088.1 | 3767987 | — | G | G | G | G | G | A |
| 14 | NW_003378088.1 | 3816341 | — | T | T | — | T | T | C |
| 15 | NW_003378088.1 | 3914549 | — | — | — | G | G | G | R |
| 16 | NW_003378088.1 | 4363281 | — | G | G | G | G | G | A |
| 17 | NW_003377992.1 | 391791 | — | C | — | C | C | C | Y |
| 18 | NW_003378010.1 | 550054 | G | G | G | G | G | G | R |
| 19 | NW_003378027.1 | 356859 | G | G | G | G | G | G | R |
| 20 | NW_003378033.1 | 211517 | C | C | C | C | C | C | Y |
| 21 | NW_003377995.1 | 360953 | — | A | A | A | A | A | R |

Note:
K represents a GT heterozygote, S represents a CG heterozygote, R represents an AG heterozygote, and Y represents a CT heterozygote; a single A or C or G or T represents a homozygote.

It can be known according to the SNP information and identification method provided by the present invention that, if T is included in the SNP information of position 1502972 of NW_003378054.1, the species to be tested is Apis mellifera sinisxinyuan; otherwise if T is not included, the species to be tested is not Apis mellifera sinisxinyuan. It can be known from Table 1 that the SNP information of the species to be tested is K, which is a GT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if C is included in the SNP information of position 292887 of NW_003378051.1, the species to be tested is Apis mellifera sinisxinyuan; otherwise if C is not included, the species to be tested is not Apis mellifera sinisxinyuan. It can be known from Table 1 that the SNP information of the species to be tested is S, which is a CG heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 1503304 of NW_003378077.1, the species to be tested is Apis mellifera sinisxinyuan; otherwise if T is not included, the species to be tested is not Apis mellifera sinisxinyuan. It can be known from Table 1 that the SNP information of the species to be tested is R, which is a GT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if A is included in the SNP information of position 420520 of NW_003378091.1, the species to be tested is Apis mellifera sinisxinyuan; otherwise if A is not included, the species to be tested is not Apis mellifera sinisxinyuan. It can be known from Table 1 that the SNP information of the species to be tested is R, which is a GT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if A is included in the SNP information of position 3914549 of NW_003378088.1, the species to be tested is Apis mellifera sinisxinyuan; otherwise if A is not included, the species to be tested is not Apis mellifera sinisxinyuan. It can be known from Table 1 that the SNP information of the species to be tested is R, which is an AG heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if A is included in the SNP information of position 550054 of NW_003378010.1, the species to be tested is Apis mellifera sinisxinyuan; otherwise if A is not included, the species to be tested is not Apis mellifera sinisxinyuan. It can be known from Table 1 that the SNP information of the species to be tested is R, which is an AG heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if A is included in the SNP information of position 356859 of NW_003378027.1, the species to be tested is Apis mellifera sinisxinyuan; otherwise if A is not included, the species to be tested is not Apis mellifera sinisxinyuan. It can be known from Table 1 that the SNP information of the species to be tested is R, which is an AG heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if G is included in the SNP information of position 360953 of NW_003377995.1, the species to be tested is Apis mellifera sinisxinyuan; otherwise if G is not included, the species to be tested is not Apis mellifera sinisxinyuan. It can be known from Table 1 that the SNP information of the species to be tested is R, which is a GT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 935827 of NW_003378074.1, the species to be tested is Apis mellifera sinisxinyuan; otherwise if T is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if C is included in the SNP information of position 512953 of NW_003378051.1, the species to be tested is Apis mellifera sinisxinyuan; otherwise if C is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 693133 of NW_003378131.1, the species to be tested is Apis mellifera sinisxinyuan; otherwise if T is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 860330 of NW_003378131.1, the species to be tested is Apis mellifera sinisxinyuan; otherwise if T is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 2595617 of NW_003378085.1, the species to be tested is Apis mellifera sinisxinyuan; otherwise if T is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 391791 of NW_003377992.1, the species to be tested is Apis mellifera sinisxinyuan; otherwise if T is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

It can be known according to the SNP information and identification method provided by the present invention that, if T is included in the SNP information of position 211517 of NW_003378033.1, the species to be tested is Apis mellifera sinisxinyuan; otherwise if T is not included, the species to be tested is not Apis mellifera sinisxinyuan. It can be known from Table 1 that the SNP information of the species to be tested is Y, which is a CT heterozygote, and therefore it is Apis mellifera sinisxinyuan and can be distinguished from other species. Accordingly, the detection method provided by the present invention is of feasibility and accurateness.

Likewise, the feasibility and accurateness of the SNP information and identification method provided by the present invention can be well demonstrated according to the experimental results of Table 1 about.

### Comparative Example 1

There are two major methods in the prior art, one is morphological identification, and the other is mitochondrial DNA polymorphism analysis.

(1) Morphological Identification of Honey Bees:

Indicators and method for morphological determination:

a. The proboscis length, and the chromaticity of upper labium and labium base:

The head of a honey bee was removed and the chromaticity of upper labium and labium base was observed. The proboscis, which includes three parts, the hind-mandible, the fore-maxilla and the glossa, was removed, spread on a slide coated with vaseline and the proboscis length was determined with a measurement system.

b. The chromaticity of the scutellum

The thorax of a bee was taken and fixed in a white wax tray. The chromaticity of the sc region, K region and B region of the scutellum was determined under a 40-fold stereomicroscope.

c. The length and width of the right fore wing, the length of the cubital vein and the angle of the wing vein angles:

The right fore wing of a honey bee was removed, flattened and laid on a slide coated with vaseline, and the length and width of the right fore wing, the cubital vein a, b, and the angle of the wing vein angles were determined with a measurement system.

d. The number of hamuli of the right hind wing:

The right hind wing of a honey bee was removed, flattened and laid on a slide, and the number of hamuli of the right hind wing was counted and recorded under a reading stereomicroscope.

e. The covering hair length on the tergite 5:

The abdomen of a honey bee was taken and the ethanol on the body surface was dried in the air, and it was laid directly under a 40-fold reading stereomicroscope to read the covering hair length on the tergite 5.

f. The length of femur, the length of tibia, the length and width of tarsus of the right hind foot of a honey bee:

The right hind foot of a bee was removed and laid on a slide coated with vaseline. The length of each indicator was determined with a measurement system respectively.

e. The length of the tergite 3 and tergite 4 of a honey bee:

The abdomen of a honey bee was dissected and the tergite 3 and tergite 4were removed and laid on a slide coated with vaseline. The length was determined with a measurement system.

f. The length of sternite 3, and the wax mirror length, the wax mirror inclined length and the wax mirror spacing of sternite 3 of a honey bee:

The sternite 3 of a honey bee was removed and the muscle tissue and wax scales on the sternite were scraped clean (be careful not to damage the wax mirror); the sternite 3 was flattened and laid on a slide coated with vaseline. The length of each indicator was determined with a measurement system respectively.

g. The length and width of the sternite 6 of a honey bee:

The sternite 6 of a honey bee was removed and the muscle tissue and wax scales were scraped clean; the sternite 6 was flattened and laid on a slide coated with vaseline, and the length and width of sternite 6 was determined with a measurement system.

h. The width of the tomentum of tergite 4 and the length form the tomentum to the bottom edge:

This indicator can be measured simultaneously with the length of tergite 4; firstly the body fluid on the tergite 4 was absorbed with absorbent paper, and then the width of the tomentum of tergite 4 and the length from the tomentum to the bottom edge were determined with a measurement system.

i. The chromaticity of tergites 2, 3 and 4:

The tergites 2, 3 and 4 were removed, and the chromaticity thereof was observed under a reading stereomicroscope.

The disadvantages of morphological identification:

a. redundant steps and cumbersome methods;

b. excessive errors;

c. inability of distinguishing between all the subspecies.

Apis mellifera sinisxinyuan and Apis mellifera mellifera can not be distinguished in morphology with the prior existing measurement indicators, however, it can be confirmed that Apis mellifera sinisxinyuan and Apis mellifera mellifera are two different subspecies with the analysis of gene recombination sequencing data.

Table 2 is the morphological identification results of Apis mellifera sinisxinyuan (sinisxinyuan), European dark bee (mellifera), Carniolan bee (carnica), Italian bee (ligustica), Caucasian bee (caucasica) and pomonella (a western honey bee discovered in Kazakhstan) with the methods of morphological identification.

TABLE 2
The morphological identification data of Apis mellifera sinisxinyuan and other major bee species

| | Xinyuan | European | | | | |
|---|---|---|---|---|---|---|
| | honey bee | dark bee | Carniolan bee | Italian bee | Caucasian bee | |
| | sinisxinyuan | mellifera | carnica | ligustica | caucasica | pomonella |
| | (N = 20) | (N = 9) | (N = 20) | (N = 12) | (N = 12) | (N = 31) |
| hairlength | 49.89 ± 2.41 | 43.47 ± 4.77 | 29.57 ± 3.74 | 27.92 ± 2.35 | 33.22 ± 1.96 | 26.85 ± 1.95 |
| proboscis | 595.00 ± 11.02 | 607.15 ± 13.12 | 640.87 ± 7.51 | 635.94 ± 7.86 | 706.64 ± 15.06 | 641.16 ± 17.96 |
| | | (N = 4) | (N = 18) | | | |
| body | 409.95 ± 7.19 | 463.02 ± 9.58 | 453.44 ± 6.25 | 436.93 ± 7.8 | 456.62 ± 7.76 | 449.49 ± 14.04 |
| size | | | | | | |
| cubital | 1.67 ± 0.16 | 1.82 ± 0.2 | 2.69 ± 0.18 | 2.52 ± 0.12 | 2.14 ± 0.12 | 2.24 ± 0.20 |
| index | | | | | | |
| stemite 6 | 68.96 ± 1.31 | 77.99 ± 2.92 | 83.81 ± 1.2 | 84.14 ± 1.49 | 82.14 ± 1.14 | 78.18 = 3.03 |
| index | | | | | | (N = 29) |
| tarsus index | 54.11 ± 1.26 | 54.86 ± 1.64 | 55.31 ± 1.37 | 55.24 ± 0.68 | 57.81 ± 0.75 | 56.78 ± 1.35 |
| pigmentation | 1.71 ± 1.68 | 2.1 ± 0.74 | 1.39 ± 0.95 | 7.87 ± 0.4 | 3.92 ± 0.27 | 5.65 ± 1.12 |
| tergite 2 | | | (N = 15) | | | |
| tomentum | 1.26 ± 0.12 | 1.63 ± 0.57 | 2.07 ± 0.26 | 2.2 ± 0.54 | 2.76 ± 0.21 | 2.05 ± 0.39 |
| index | | | | | | |

It can be seen from the results that Apis mellifera sinisxinyuan and Apis mellifera mellifera can not be distinguished with morphological identification.

### Comparative Example 2

(2) Analysis of Mitochondrial DNA Polymorphism in Honey Bees:

The different subspecies of honey bee are typically formed due to geographical isolation and ecological isolation. The isolations lead to variations in subspecies, including mtRNA variations. Such variations accumulate into different directions and the genetic diversity appears in mtRNA of each subspecies. Wherein, the most classical variation is that the length of the region between the gene of cytochrome C oxidase subunit I (COI) and the gene of cytochrome C oxidase II (COII) varies among different subspecies. Depending on the length of mRNA fragment or SNP information as the feature of a subspecies, it in turn can be considered as a genetic marker of this subspecies to be distinguished from other subspecies. For example, three main branches were formed in western honey bees in the process of evolution in accordance with the data of restriction map and sequence analysis of mtRNA. Such branching occurred about 1,000,000 years ago, wherein the first branch (Branch M) corresponds to the Western European subspecies, Apis mellifera mellifera; the second branch (Branch C) spreads from the Middle East to Italy, including Iranian bees, Caucasian bees, Greek bees, Carniolan bees and Italian bees. The third branch (Branch A) includes African honey bees and Africanized honey bees in the America, such as Tunisia bee, West Africa bee, East Africa bee, Cape bee and Kilimanjaro bee.

However, some of the subspecies cannot be categorized by mtDNA, such as Apis mellifera sinisxinyuan and Apis mellifera mellifera, which cannot be distinguished with the prior method of mtDNA sequence analysis. However, it can be confirmed that Apis mellifera sinisxinyuan and Apis mellifera mellifera are two different subspecies with the analysis of gene recombination sequencing data.

The phylogenetic tree constructed with the mitochondrial DNA sequencing data on Apis mellifera sinisxinyuan and other major bee species.

It can be seen in FIG. 2 that Apis mellifera sinisxinyuan and Apis mellifera mellifera are clustered together and can not be distinguished from each other.

The foregoing is only preferred embodiments of the present invention, it should be noted that improvements and modifications may be made by those skilled in the art without departing from the principles of the present invention, and such improvements and modifications should also to be deemed to be within the protection scope of the present invention.

1. A SNP marker, selected from one or more of:

| | | |
|---|---|---|
| NW_003378074.1 | Position 935827 | SNP information T; |
| NW_003378158.1 | Position 846767 | SNP information A; |
| NW_003378051.1 | Position 292887 | SNP information C; |
| NW_003378051.1 | Position 512953 | SNP information C; |
| NW_003378077.1 | Position 1503304 | SNP information T; |
| NW_003378131.1 | Position 693133 | SNP information T; |
| NW_003378131.1 | Position 860330 | SNP information T; |
| NW_003378091.1 | Position 420520 | SNP information A; |
| NW_003378085.1 | Position 1765707 | SNP information A; |
| NW_003378085.1 | Position 1775747 | SNP information T; |
| NW_003378085.1 | Position 2595617 | SNP information T; |
| NW_003378054.1 | Position 1502972 | SNP information T; |
| NW_003378088.1 | Position 3767987 | SNP information A; |
| NW_003378088.1 | Position 3816341 | SNP information C; |
| NW_003378088.1 | Position 3914549 | SNP information A; |
| NW_003378088.1 | Position 4363281 | SNP information A; |
| NW_003377992.1 | Position 391791 | SNP information T; |
| NW_003378010.1 | Position 550054 | SNP information A; |
| NW_003378027.1 | Position 356859 | SNP information A; |
| NW_003378033.1 | Position 211517 | SNP information T; |
| NW_003377995.1 | Position 360953 | SNP information G. |

2. A method for use in identification of a species by using the SNP marker according to claim 1, wherein the species is Apis mellifera sinisxinyuan.

3. A method for use in molecular marker-assisted breeding of Apis mellifera sinisxinyuan by using the SNP marker according to claim 1.

4. A method for use in adaptive evolution of species by using the SNP marker according to claim 1.

5. A method for identifying Apis mellifera sinisxinyuan with the SNP marker according to claim 1, comprising:
step 1: obtaining the DNA of a species to be tested, amplifying with a primer pair for detecting the SNP marker of claim 1 to obtain an amplification product;
step 2: subjecting the amplification product obtained in Step 1 to single nucleotide polymorphism detection, wherein the detection criterion is as follows:

| | | |
|---|---|---|
| NW_003378074.1 | Position 935827 | SNP information T; |
| NW_003378158.1 | Position 846767 | SNP information A; |
| NW_003378051.1 | Position 292887 | SNP information C; |
| NW_003378051.1 | Position 512953 | SNP information C; |
| NW_003378077.1 | Position 1503304 | SNP information T; |
| NW_003378131.1 | Position 693133 | SNP information T; |
| | | |
|---|---|---|
| NW_003378131.1 | Position 860330 | SNP information T; |
| NW_003378091.1 | Position 420520 | SNP information A; |
| NW_003378085.1 | Position 4765707 | SNP information A; |
| NW_003378085.1 | Position 1775747 | SNP information T; |
| NW_003378085.1 | Position 2505617 | SNP information T; |
| NW_003378054.1 | Position 1502972 | SNP information T; |
| NW_003378088.1 | Position 3767987 | SNP information A; |
| NW_003378088.1 | Position 3816341 | SNP information C; |
| NW_003378088.1 | Position 3914549 | SNP information A; |
| NW_003378088.1 | Position 4363281 | SNP information A; |
| NW_003377992.1 | Position 391791 | SNP information T; |
| NW_003378010.1 | Position 550054 | SNP information A; |
| NW_003378027.1 | Position 356859 | SNP information A; |
| NW_003378033.1 | Position 211517 | SNP information T; |
| | | |
|---|---|---|
| NW_003377995.1 | Position 360953 | SNP information G. |

6. The method according to claim 5, wherein if the species to be tested satisfies 95% or more of the detection criterion, the species to be tested is Apis mellifera sinisxinyuan; if the species to be tested does not satisfy at least two of the detection criteria, the species to be tested is not Apis mellifera sinisxinyuan.

7. The method according to claim 5, wherein if the species to be tested satisfies any 20 or 21 SNPs of the 1st to 21st SNPs, it is Apis mellifera sinisxinyuan.
